(19) United States
(12) Patent Application Publication
(10) Pub. No.: US 2020/0269256 A1
(43) Pub. Date: Aug. 27, 2020

(54) Syringe Holder

(71) Applicant: Stephen S. Ho, Houston, TX (US)

(72) Inventor: Stephen S. Ho, Houston, TX (US)

(21) Appl. No.: 16/282,292

(22) Filed: Feb. 21, 2019

(51) Int. Cl. B04B 5/04; A61K 9/00; A61K 35/19; B01L 3/00; A61M 1/36; A61M 1/02

(52) U.S. Cl. CPC B04B 5/0421; A61K 9/0019; A61K 35/19; A61K 35/16; A61M 1/3693; A61M 1/029; B01L 3/5021

(57) ABSTRACT

A syringe holder for holding a syringe in a centrifuge, the syringe having a plunger-end and a nipple-end, the syringe holder holding the syringe nipple-end upwardly, comprising: a cup having a cup seat, a wall, a bore, a mouth, and a support, the support supporting the cup in the centrifuge; a lower support having a upper surface and a lower surface, the lower support sized to slide into the cup and the lower support supporting the plunger-end of the syringe; an upper support having lower section and an upper section with a lip, and having a channel therethrough, the lip having a outer diameter larger than the inner diameter of the mouth of the cup, the channel therethrough co-axial with the lower support and the bore of the cup, the lower section having an outer diameter that is less than the inner diameter of the bore of the of the cup and sized to permit the upper support to slide at least partially into the cup and the lip to contact the mouth of the cup and support the upper support in the cup; whereby the upper support and the lower support support the syringe with the needle-end upwardly in the cup during centrifuging.

### STATEMENT REGARDING FEDERALLY SPONSORED RESEARCH

Not Applicable.

### PRIORITY CLAIM TO EARLIER APPLICATIONS

None

### TECHNICAL FIELD OF THE INVENTION

The technical field of this disclosure relates to the collection and isolation of cells collected from a patient.

### BACKGROUND OF THE INVENTION

Collection and isolation of a person's own cells and the reintroduction of the isolated cells can have beneficial therapeutic results. For example, platelet-rich-plasma (PRP), stem cells and fat can be collected from a patient, isolated and then returned to the patient's own body to provide beneficial therapeutic results.

In order to obtain samples of stem cells or PRP, a sample of whole blood or fat is collected from the patient. Similarly, to obtain fat for use in the patient, a fat grafting procedure transfers fat from areas where the patient has excess fat, such as the outer thigh, abdomen, or buttocks and injects it into areas that may be lacking volume, i.e. volume deficient areas.

Typical difficulties with these types of processes include cell necrosis. Thus, it is important to have processes that both collect and isolate the desired cells and also mitigates that risk that the cells that have been isolated die or are damaged, i.e. cell necrosis.

FIG. 1A illustrates a patient having cell sample(s) collected.

FIG. 1B illustrates a perspective view of a conventional centrifuge centrifuging a sample that has been collected using a conventional syringe. The sample is illustrated on the left as being transferred into a conventional test tube and then placed in the centrifuge or the sample could be retained in the syringe and placed in the centrifuge. This is undesirable because the centrifuge cup has a larger diameter than the test tube or conventional syringe. As a result, during centrifuging, the test tube/syringe moves within the cup. This is undesirable because this movement within the cup can increase cell necrosis. As also illustrated in FIG. 1B, during conventional centrifuging syringe is positioned nipple-end down. This is undesirable because the conventional syringe is more prone to leaking when faced downwardly. Leaking and spilling may also occur during centrifuging. In addition, if a sample is transferred from a collection syringe into a test tube for centrifuging (test tube illustrated on left of centrifuge), spillage may occur during the transfer of the sample to the test tube and, potentially, when the sample is transferred back to the syringe.

An alternative embodiment of the syringe could be the cell collection apparatus illustrated in U.S. Utility Patent application Ser. No. 15/726,981, filed Oct. 6, 2017, for An Apparatus and Method for Isolating Cells, and also to International Application No. PCT/US2018/054713, filed Oct. 5, 2018 with the United States Patent Office as the Receiving Office, for An Apparatus and Method of Isolating Cells. Applicant's earlier filings are hereby incorporated by reference as if fully set forth herein.

As disclosed and taught in the following materials, a variety of methods and apparatus/systems may be used in the isolation of stem cells, platelets and fat cells.

### Background

Methods of isolating the patient's cells may also provide therapeutic benefit.

For example, U.S. Pat. No. 6,398,972, “Method of Producing Platelet Rich Plasma and/or Platelet Concentration,” issued to Blasetti et al., on Jun. 4, 2002, discloses platelet rich plasma and/or platelet concentrate prepared by placing whole blood in a first chamber of a sterile processing disposable having two chambers. The processing disposable is subjected to a first centrifugation to separate red blood cells, and the resulting platelet rich plasma super-natant is decanted to the second chamber. The processing disposable is subjected to a second centrifugation to concentrate platelets. A volume of platelet poor plasma supernatant in the second chamber is removed, and the platelets are re-suspended in the remaining plasma. The second chamber may contain anticoagulant to preclude aggregation of the platelets.

As another example, International Application published under the Patent Cooperation Treaty under International Publication No. WO2005105121A1 on Nov. 10, 2005, by Meury et al., discloses the use of the content of platelets or platelet rich plasma (PRP) obtained by disruption of their membranes for the preparation of an agent for the treatment of bone, cartilage or skin.

Another example, U.S. Patent Application Publication No. 2007/0280959, published on Dec. 6, 2007, by Meury et al., discloses a new use for the contents of platelets or platelet rich plasma (PRP) obtained by disruption of their membranes for the preparation of an agent for the treatment of bone, cartilage or skin.

Another example, European Patent Office Publication EP2628484A1, published on Aug. 21, 2013, by Agut Sanchez, et al., discloses a pharmaceutical composition for oral administration comprising platelet-rich plasma and uses thereof. The invention also relates to orally administered cosmetic compositions and nutritional compositions comprising platelet-rich plasma.

Yet another example, U.S. Patent Application Publication 2015/0044179A1, by Masanori Saeki, discloses a cell preparation useful for treatment of diseases in bone joints and repairing muscles which have been degenerated or damaged. And, also discloses a cell preparation for treatment of diseases in bone joints which comprises a fat cell, and a cell preparation for repairing muscles which comprises a fat cell and a mesodermal stem cell.

### SUMMARY OF THE INVENTION

A syringe holder for holding a syringe in a centrifuge, the syringe having a plunger-end and a nipple-end, the syringe holder holding the syringe nipple-end upwardly, comprising: a cup having a cup seat, a wall, a bore, a mouth, and a support, the support supporting the cup in the centrifuge; a lower support having a upper surface and a lower surface, the lower support sized to slide into the cup and the lower support supporting the plunger-end of the syringe; an upper support having lower section and an upper section with a lip, and having a channel therethrough, the lip having a outer diameter larger than the inner diameter of the mouth of the cup, the channel therethrough co-axial with the lower support and the bore of the cup, the lower section having an outer diameter that is less than the inner diameter of the bore of the of the cup and sized to permit the upper support to slide at least partially into the cup and the lip to contact the mouth of the cup and support the upper support in the cup; whereby the upper support and the lower support support the syringe with the needle-end upwardly in the cup during centrifuging.

These and other embodiments will be more fully appreciated from the description below.

### BRIEF DESCRIPTION OF THE DRAWINGS

FIG. 1A illustrates a patient having cell sample(s) collected.

FIG. 1B illustrates a perspective view of a conventional centrifuge centrifuging a sample that has been collected using a conventional syringe and also a sample in which the cell sample has been transferred to a test tube.

FIG. 2A illustrates a perspective view of a syringe holder.

FIG. 2B illustrates an exploded perspective view of the syringe holder of FIG. 2A.

FIG. 3 illustrates an environmental perspective view of the syringe holder with a syringe.

FIG. 4 illustrates a front view of the syringe holder with a syringe. The rear view of the syringe holder with a syringe is a mirror image. The syringe is shown in dashed lines.

FIG. 5 illustrates a right side view of the syringe holder with a syringe. The left side view of the syringe holder with a syringe is a mirror image of FIG. 5.

FIG. 6 illustrates a top view of the syringe holder.

FIG. 7 illustrates a bottom view of the syringe holder.

FIG. 8 illustrates a front view of a cup of the syringe holder. The rear view of the cup of the syringe holder is a mirror image of the front view.

FIG. 9 illustrates a right side view of the cup of the syringe holder.

FIG. 10 illustrates a top view of the cup of the syringe holder.

FIG. 11 illustrates a bottom view of the cup of the syringe holder.

FIG. 12 illustrates a front view of an upper support. The rear view, right side view, and left side view of the upper support are mirror images.

FIG. 13 illustrates a top view of the upper support.

FIG. 14 illustrates a bottom view of the upper support.

FIG. 15 illustrates a front view of a lower support. The rear view, right side view, and left side view of the lower support are mirror image of the front view.

FIG. 16 illustrates a top view of the lower support.

FIG. 17 illustrates a bottom view of the lower support.

FIG. 18 illustrates a perspective view of centrifuging the syringe holder holding the syringe.

### DESCRIPTION OF THE PREFERRED EMBODIMENTS

Corresponding reference numbers indicate corresponding parts throughout the several views of the drawings and specification.

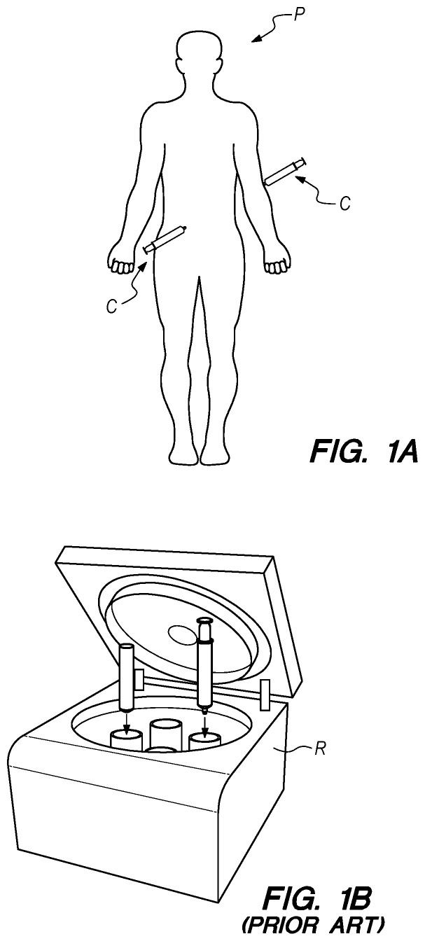

FIG. 1A illustrates a patient P having her cells collected using a syringe C from various different portions of her body. As illustrated in FIG. 1A, syringe C could be used to collect cells by drawing whole blood from patient P's veins (also referred to as “venipuncture.”). Typically, a needle is used for venipuncture. Similarly, syringe C could also be used to collect a tissue sample of patient P's fat cells. Typically, a cannula is used for fat collection. As illustrated in FIG. 1A, fat cells could be collected from patient P's abdomen or others areas of the body with sufficient fatty deposits. Patient P's tissue sample could then be centrifuged thereby isolating different cell types. As an example, a whole blood sample could be collected from a patient. The whole blood sample could then be centrifuged thereby separating the whole blood sample into its component parts; such as plasma and platelets. Similarly, human fat tissue can be collected and centrifuge to isolate different cells types, such as stem cells and other cells. As discussed above, it can be beneficial for a patient to have a tissue sample collected from her body, certain desirable cells isolated, and the desirable cells re-introduced into the patient's body. For example, it is believed that stem cells isolated from a patient can be re-injected back into a patient to speed healing or provide other health advantages. As an example, the re-injection of a patient's own fatty cells into the patient's cheeks can improve a patient's appearance and provide a more youthful appearance. A variety of health and cosmetic benefits can be achieved by re-injecting a patient's own cells. However, in order to improve these results, it is desirable to isolate the cells that are beneficial. Phrased differently, certain cells may have beneficial effects when re-injected and encourage repair and regeneration of the patient's body. As an example, re-injection of a patient's stem cells is believed to improve healing of the patient's injured joints. It is beneficial to re-introduce the patient's own cells because the patient will not have an autoimmune response to his or her own cells. Phrased differently, cells collected from another person would result in an autoimmune response from the patient and destruction of the foreign cells. Thus, re-introduction of the patient's own cells is highly desirable.

FIG. 1B illustrates a view of a conventional centrifuge centrifuging a sample that has been collected using a conventional syringe, in the example seen on the left of the centrifuge; the sample is illustrated as being transferred into a conventional test tube for centrifuging. Alternatively, as seen on the right side of the centrifuge, the sample could be retained in the syringe. FIG. 1B illustrates that if the sample is retained in the syringe during centrifuging, the syringe is placed such that the needle-end of the syringe is placed downwardly into the centrifuge. This is undesirable because the conventional syringe, under certain circumstances, is more prone to leaking when faced downwardly and also may move or shift within the centrifuge because the centrifuge holder has a larger diameter than the syringe.

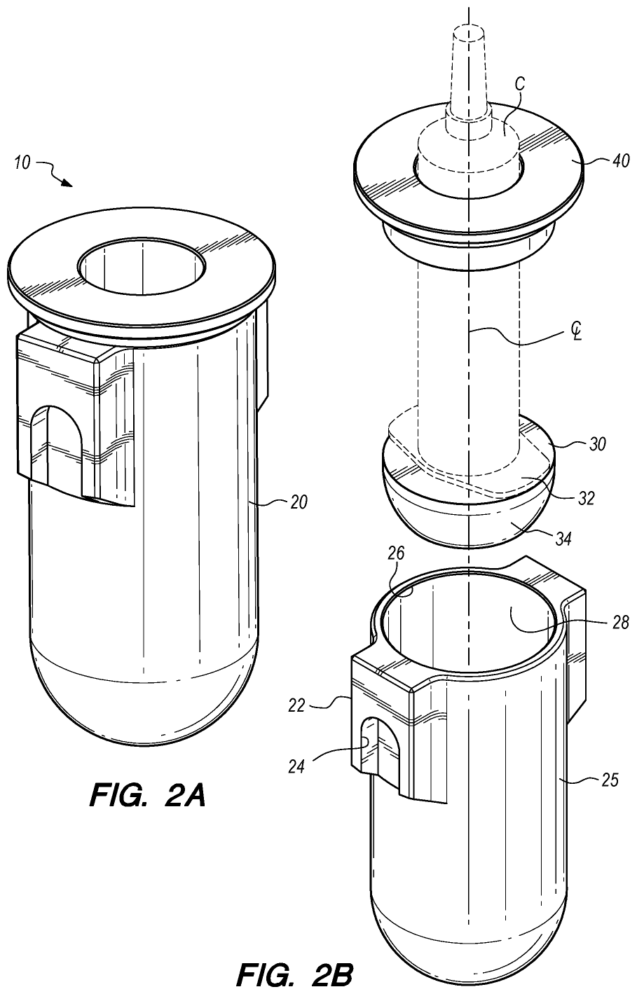

FIG. 2A illustrates a perspective view of a syringe holder 10,

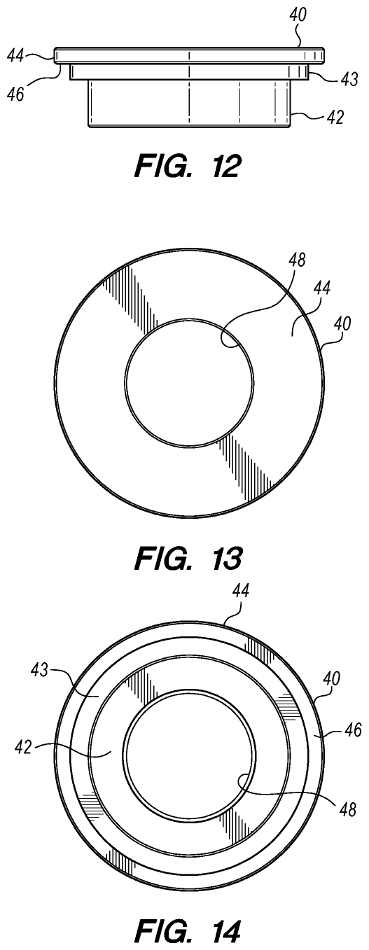

FIG. 2B illustrates an exploded view of the syringe holder 10. Syringe holder 10 comprises a cup 20, a lower support 30, and an upper support 40. A syringe or tube C containing a cell sample is illustrated in dashed lines. Preferably, syringe C is the cell collection apparatus illustrated in U.S. Utility patent application Ser. No. 15/726,981, filed Oct. 6, 2017, for An Apparatus and Method for Isolating Cells, and also in International Application No. PCT/US2018/054713, filed Oct. 5, 2018 with the United States Patent Office as the Receiving Office, for An Apparatus and Method of Isolating Cells that are hereby incorporated by reference as if fully set forth herein. The inventor of this application Dr. Stephen Ho is also the inventor and applicant of the two earlier applications. FIG. 2B also illustrates that cup 20 has supports 22, slides 24, wall 25, mouth 26, and bore 28. Mouth 26 is preferably circular. However, other shapes, such as oval may be useful. FIG. 2B also illustrates that lower support 30 has upper surface 32, lower surface 34, and seat 36. FIG. 2B also illustrates that upper support 40 has upper section 42, lower section 44, lip 46, and channel 48. Intermediate section 43 is not clearly visible in FIG. 2B, but can be clearly seen in FIGS. 12 and 14. FIG. 2B also illustrates that the longitudinal axis of lower support 30 and upper support 40 coincide. Phrased differently, the longitudinal (center line) axis of cup 20, lower support 30, upper support 40, and syringe C are co-axial.

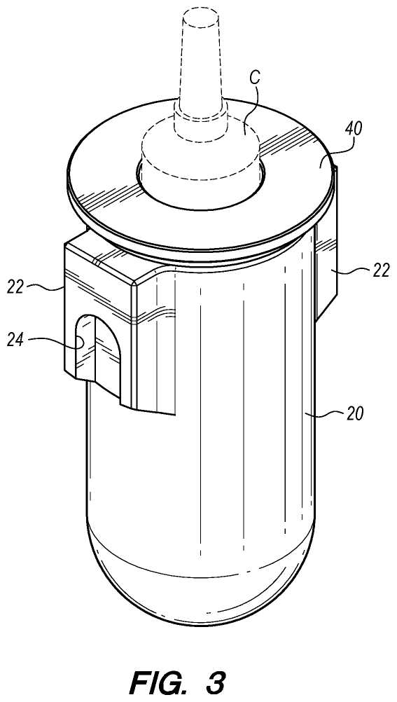

FIG. 3 illustrates an environmental perspective view of the syringe holder 10 with syringe C and cup 20 and upper support 40. Syringe C is shown in dashed lines. FIG. 3 also illustrates supports 22.

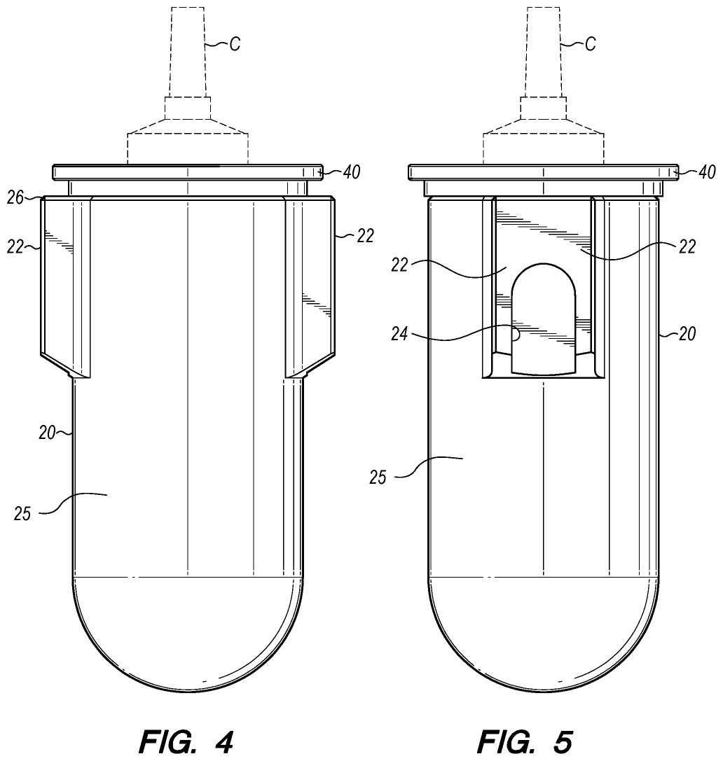

FIG. 4 illustrates a front view of the syringe holder 10 with syringe C. Syringe C is shown in dashed lines. Cup 20 comprises supports 22, slides 24, wall 25, mouth 26, and bore 28. Preferably, supports 22 are opposing.

FIG. 5 illustrates a right side view of the syringe holder 10 in use with syringe C. The left side view of the syringe holder 10 in use with syringe C is a mirror image of FIG. 5. Support 22 and slide 24 are used to support cup 20 in a centrifuge R. Preferably, a portion of centrifuge R slides into slide 24 and slidably retains syringe holder 10 within centrifuge R.

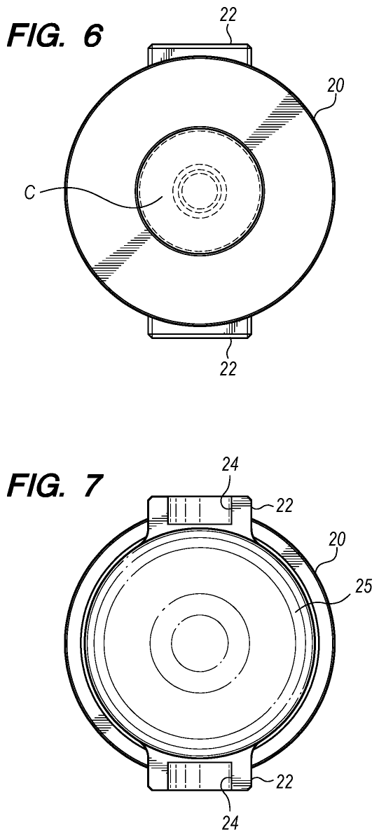

FIG. 6 illustrates a top view of the syringe holder 10. Supports 22 are also illustrated in FIG. 6.

FIG. 7 illustrates a bottom view of the syringe holder 10. Supports 22, slides 24, and wall 25 are also illustrated in FIG. 7.

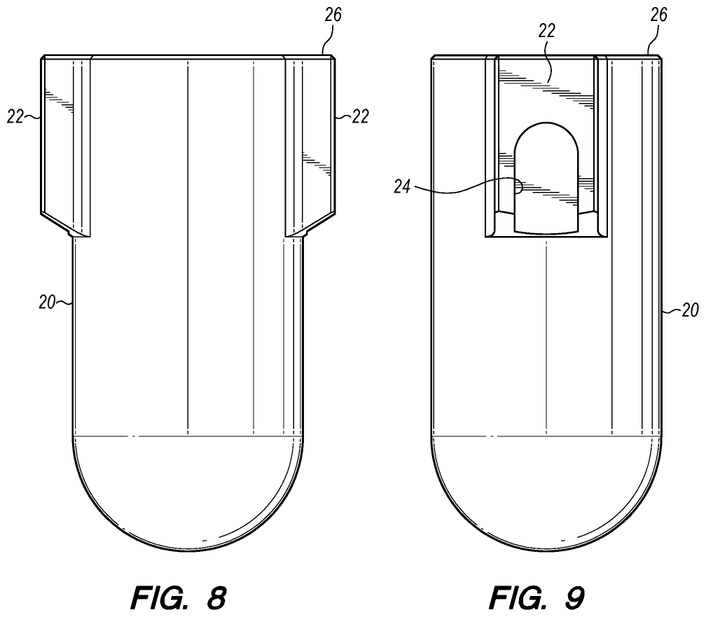

FIG. 8 illustrates a front view of the cup 20 of syringe holder 10. The back view of cup 20 of syringe holder 10 is the mirror image of the front view.

FIG. 8 illustrates cup 20, supports 22, and mouth 26.

FIG. 9 illustrates a right side view of the syringe holder 10. FIG. 9 also illustrates supports 22, slide 24, and mouth 26.

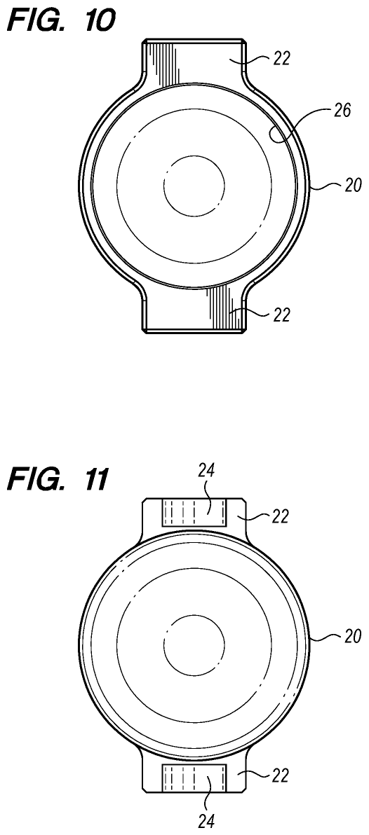

FIG. 10 illustrates a top view of the cup of the syringe holder 10. FIG. 10 also illustrates supports 22, wall 25, and mouth 26.

FIG. 11 illustrates a bottom view of the cup of the syringe holder 10. FIG. 11 also illustrates supports 22 and slides 24.

FIG. 12 illustrates a front view of upper support 40. Upper support 40 comprises lower section 42, intermediate section 43, upper section 44, lip 46, and bore 48. Preferably, upper support 40 includes intermediate section 43. However, intermediate section 43 could be omitted.

FIG. 13 illustrates a top view of upper support 40. FIG. 13 also illustrates upper section 44 and bore 48.

FIG. 14 illustrates a bottom view of the upper support 40. Bore 48 and lip 46 are more clearly seen in FIG. 14. As discussed above, lip 46 extends over the top of mouth 26 of cup 20, such that upper support 40 sits in cup 20. FIG. 14 also illustrates lower section 42, intermediate section 43, and upper section 44.

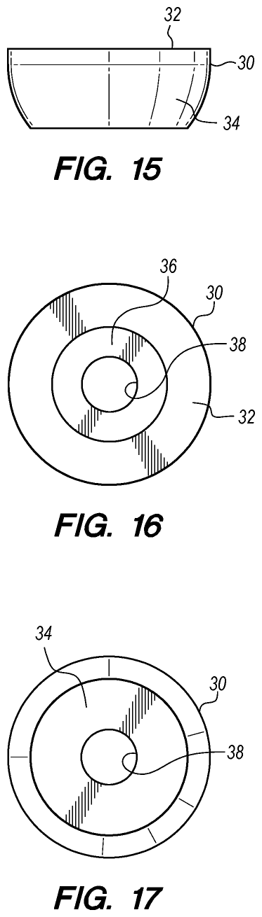

FIG. 15 illustrates a front view of the lower support 30. Upper surface 32 and lower surface 34 of lower support 30 are also illustrated in FIG. 15.

FIG. 16 illustrates a top view of the lower support 30. Lower support 30 comprises upper surface 32, lower surface 34 and seat 36. Preferably, bore 38 is disposed therethrough lower support 30. Bore 38 may be omitted if preferred. Bore 36 minimizes the pressure difference that can make it more difficult to slidably remove lower support 30 from cup 20.

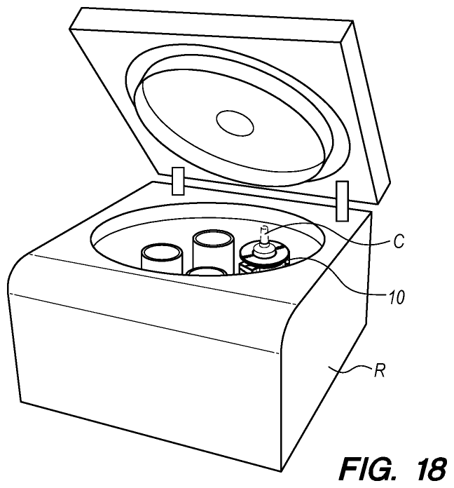

FIG. 17 illustrates a bottom view of the lower support member. FIG. 17 also illustrates lower surface 34 and bore 38.

Upper support 40 and lower support 30 may be formed from a variety of materials, such as a plastic, a thermo-resilient plastic, or a ceramic.

FIG. 18 illustrates a perspective view of a centrifuge R centrifuging a syringe holder 10 holding a syringe C. To emphasize, unlike FIG. 1B, syringe C is positioned with the nipple-end upwardly. Typically, a centrifuge R would centrifuge syringe holders 10 such that they are balanced in opposing pairs. In FIG. 18, single syringe holder 10 is shown by itself. However, it is expected that pairs of opposing pairs of syringe holders 10 will be employed. FIG. 18's centrifuge R could centrifuge four syringe holders 10 holding four syringes C. Depending on the positions available, centrifuge R could centrifuge more or less centrifuge holders 10 holding syringes C.

While the invention has been illustrated and described in detail in the drawings and description, the same is to be considered as an illustration and is not limited to the exact embodiments shown and described. All equivalents, changes and modifications that come within the spirit of the invention are also protected by the claims that are set forth below.

I claim:

1. A syringe holder for holding a syringe in a centrifuge, the syringe having a plunger-end and a nipple-end, the syringe holder holding the syringe nipple-end upwardly, comprising:
a cup having a cup seat, a wall, a bore, a mouth, and a support, the support supporting the cup in the centrifuge;
a lower support having a upper surface and a lower surface, the lower support sized to slide into the cup and the lower support supporting the plunger-end of the syringe;
an upper support having lower section and an upper section with a lip, and having a channel therethrough, the lip having a outer diameter larger than the inner diameter of the mouth of the cup, the channel therethrough co-axial with the lower support and the bore of the cup, the lower section having an outer diameter that is less than the inner diameter of the bore of the of the cup and sized to permit the upper support to slide at least partially into the cup and the lip to contact the mouth of the cup and support the upper support in the cup;
whereby the upper support and the lower support the syringe with the needle-end upwardly in the cup during centrifuging.

2. The syringe holder of claim 1, further comprising: an intermediate section between the upper section and the lower section of the upper support.

3. The syringe holder of claim 2, further comprising: the lower support having a seat.

4. The syringe holder of claim 3, the lower section having a bore therethrough.

5. The syringe holder of claim 4, further comprising a slide, wherein a portion of the centrifuge is slidably received by the slide and supports the cup within the centrifuge.

6. The syringe holder of claim 5, further comprising: a centrifuge.

7. The syringe holder of claim 6, wherein the centrifuge can centrifuge one or more syringes.

8. The syringe holder of claim 7, further comprising: forming the upper support or the lower support from a thermo-resilient plastic.

9. The syringe holder of claim 7, further comprising: forming the upper support or the lower support from a plastic, a thermo-resilient plastic or a ceramic material.

10. The syringe holder of claim 7, further comprising, the mouth of the cup is circular or oval.

11. A method of centrifuging a syringe, the syringe having a plunger-end and a nipple-end, the syringe holder holding the syringe nipple-end upwardly, comprising:
selecting a syringe holder having a cup having a cup seat, a wall, a bore, a mouth, and a support, the support supporting the cup in the centrifuge; a lower support having a upper surface and a lower surface, the lower support sized to slide into the cup and the lower support supporting the plunger-end of the syringe; an upper support having lower section and an upper section with a lip, and having a channel therethrough, the lip having a outer diameter larger than the inner diameter of the mouth of the cup, the channel therethrough co-axial with the lower support and the bore of the cup, the lower section having an outer diameter that is less than the inner diameter of the bore of the of the cup and sized to permit the upper support to slide at least partially into the cup and the lip to contact the mouth of the cup and support the upper support in the cup; and, whereby the upper support and the lower support support the syringe with the needle-end upwardly in the cup during centrifuging.

12. The syringe holder of claim 11, further comprising: an intermediate section between the upper section and the lower section of the upper support.

13. The syringe holder of claim 12, further comprising: the lower support having a seat.

14. The syringe holder of claim 13, further comprising: the lower section having a bore therethrough.

15. The syringe holder of claim 14, further comprising a slide, wherein a portion of the centrifuge is slidably received by the slide and supports the cup within the centrifuge.

16. The syringe holder of claim 15, further comprising: a centrifuge.

17. The syringe holder of claim 16, wherein the centrifuge can centrifuge one or more syringes.

18. The syringe holder of claim 17, further comprising: forming the upper support or the lower support from a thermo-resilient plastic.

19. The syringe holder of claim 17, further comprising: forming the upper support or the lower support from a plastic, a thermo-resilient plastic or a ceramic material.

20. The syringe holder of claim 17, further comprising, the mouth of the cup is circular or oval.
